(19) United States
(12) Patent Application Publication
(10) Pub. No.: US 2015/0282847 A1
(43) Pub. Date: Oct. 8, 2015

(54) Method of Positioning Pedicle Screws and Spinal Rods and Apparatuses for the Same

(71) Applicants: Charles R. Gordon, Tyler, TX (US); Andy J. Redmond, Tyler, TX (US)

(72) Inventors: Charles R. Gordon, Tyler, TX (US); Andy J. Redmond, Tyler, TX (US)

(21) Appl. No.: 14/438,185

(22) PCT Filed: Oct. 23, 2013
(86) PCT No.: PCT/US2013/066400

§ 371 Date: Apr. 23, 2015

Related U.S. Application Data
(60) Provisional application No. 61/717,356, filed on Oct. 23, 2012.

(51) Int. Cl. A61B 17/70

(52) U.S. Cl. CPC A61B 17/7083

(57) ABSTRACT

Systems and methods for placing orthopedic spine fixation hardware are provided.

This application claims the benefit of priority to U.S. Provisional Patent Application Ser. No. 61/717,356, filed Oct. 23, 2012, hereby incorporated by reference in its entirety.

### FIELD OF THE INVENTION

The present invention relates generally to systems for the placement of orthopedic spine fixation devices. More specifically, the present invention also relates to systems for the minimally invasive placement of orthopedic spine fixation devices.

### BACKGROUND

Spine fixation devices are typically used by medical practitioners to fix one or more spine segments to one another for purposes of stabilizing the spine, minimizing spinal joint movement, and/or to immobilize sections of the spine to allow the healing of spinal fusions. Numerous spine fixation devices have been devised for various spine fixation needs. By way of example, various spine fixation devices have been devised for anterior placement, posterior placement, for cervical placement and for lumbar placement among others. One popular method of performing spine fixations, and/or fusions involves the placement of screws through one or more of the pedicles joining the dorsal spinal elements with the vertebral bodies on each of the spine segments to be stabilized. Rigid ties, commonly metal rods, are then rigidly affixed to the screws such that they span the distance between spine segments. Unfortunately, exposure of spine segments to the degree required to place pedicle screws and accompanying ties often requires extensive dissection and can result in significant recovery time and post-operative pain. Another risk of pedicle screw placement involves the difficulty in properly positioning pedicle screws to avoid penetration of the pedicle cortex and accompanying injuries during insertion of the screws.

### SUMMARY

The present disclosure includes embodiments of systems and methods for placing orthopedic spine fixation hardware. Some embodiments of the present invention are usable in less-invasive, or minimally invasive procedures.

Systems and methods are disclosed for placing orthopedic spine fixation hardware. In some embodiments, the systems of the present invention comprise an elongated guide; a tie; an orthopedic fastener having at least one tie-in point, a driver coupling, and a through-bore along the longitudinal axis of the fastener, which though-bore defines a lumen. The lumen may be sized to permit movement of the elongated guide through the lumen. Included may be a tie guide comprising at least one elongated section coupled to a girdling section may also be provided, the girdling section having an aperture sized to permit movement of the elongated guide through the aperture. A driver configured to engage the driver coupling when the elongated guide is disposed through both the lumen and the girdling section where the tie is coupled to the tie guide and the tie guide is configured to be capable of pulling at least a portion of the tie in to a position substantially near the tie-in-point when the elongated guide is removed from the girdling section.

In some embodiments the driver may be additionally configured to move the girdling section into a position substantially near the tie-in-point. In further embodiments, the fastener may additionally comprise a locking mechanism which may be configured to engage with the fastener and to couple at least a portion of the tie to the tie-in-point. In some embodiments the locking mechanism is capable of being either partially engaged or fully engaged, or not engaged.

In some embodiments the tie guide is configured to be capable of pulling at least a portion of the tie into a position substantially near the tie-in point when the locking mechanism is partially engaged. The tie guide may also pull at least a portion of the tie into a position substantially near multiple tie-in-points when the locking mechanism is partially engaged. In some embodiments, fully engaging the locking mechanism couples at least a portion of the tie to the tie-in-point. The locking mechanism may also comprise a through-bore defining a passage sized to permit movement of the elongated guide through the passage. The through bore of the locking mechanism may be disposed along the longitudinal axis of the fastener.

In some embodiments the driver may comprise a through-bore sized to permit movement of the first guidance structure through the driver lumen. The locking mechanism may comprise a driver coupling capable of being engaged by the driver when the elongated guide is disposed through the passage in the fastener. A driver, in some embodiments may comprise a through bore defining a driver-lumen sized to permit movement of the elongated guide through the driver lumen.

In embodiments comprising a tie-guide with one or more girdling sections, the girdling section may be substantially ring shaped. In some embodiments the tie-guide is selectively decouplable from the tie. Embodiments of the present invention comprise more than one, and/or matching numbers of elements of the embodiments.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 shows view illustrating two adjacent spinal segments with emplaced screw guides according to one embodiment of the present invention.

FIG. 2 shows a view illustrating two adjacent spinal segments with emplaced guides and cannulated screws according to one embodiment of the present invention.

FIG. 3 shows the elements of FIG. 2 with a tie rod coupled to a partially emplaced tie guide.

FIG. 4 shows a tie guide emplaced on screw guides.

FIG. 5 shows partial emplacement of locking mechanisms and driver, tie guides are moved in to a position substantially near tie-in-points of pedicle screws.

FIG. 6 partially emplaced locking mechanisms, screw guides have been removed.

FIG. 7 shows tie guide having pulled tie rod into position near tie-in-points; locking mechanisms are fully engaged.

FIG. 8 shows fully emplaced spine fixation hardware including pedicle screws, tie rod, and locking mechanisms; tie guide has been decoupled from tie rod.

### DETAILED DESCRIPTION OF THE INVENTION

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system or apparatus that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a structure (e.g., a component of an apparatus) that is configured in a certain way is configured in at least that way, but it can also be configured in other ways than those specifically described.

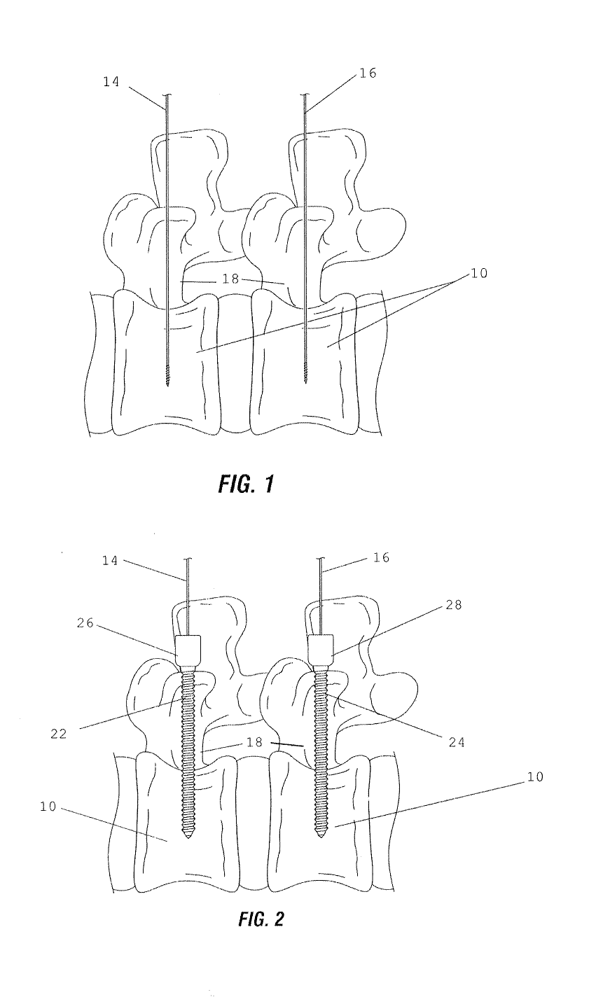

Referring now to the drawings, and more particularly to FIG. 1, shown there are adjacent spinal segments 10 into which elongated guide pins or wires 14, 16 have been inserted starting posteriorly extending through the pedicles 18 and ending within the vertebral bodies. Guide pins 14, 16 may be threaded, or partially threaded according to some embodiments of the present invention to assist insertion of the rods into their positions. According to some embodiments of the invention, the guides 14, 16 may be Kirschner wires (“K-Wires”) but may be any other types of rigid or semi-rigid elongated guides. In some embodiments, the guides are preferably stainless steel, metallic, or manufactured from radio-opaque materials. The use of radio-opaque materials is beneficial according to some embodiments due to the ability of such materials to be easily distinguished from tissues and bone under fluoroscopy and other imaging techniques that may be used to assist, or guide medical personnel during placement of the guides.

FIG. 2 shows the same adjacent spinal segments 10 with emplaced guide pins 14, 16. Additionally shown are orthopedic fasteners 22, 24 which have been driven into the spinal segments along the path of the guide pins 14, 16. According to some embodiments, the fasteners may be threaded, or partially threaded pedicle screws commonly used with spinal fixation hardware but other fasteners are contemplated within the scope of the invention. The distal ends of the fasteners 22, 24 are embedded within the vertebral body of the spine segments 10 with the shaft extending from the distal end, through the pedicles 18 to the proximal end of the fasteners. Near the proximal end of the fasteners are tie-in-points 26, 28. In the embodiment depicted, the tie-in point 26 is part of an element protruding posteriorly from the fasteners that also accepts a threaded locking mechanism portion of the fastener meant to securely couple a tie-rod to the fastener. According to some embodiments, the tie-in point is a cup shaped rosette but may be any structure suitable for securely coupling a tie to the fastener. The fastener may be a unitary body incorporating the tie-in-point, or it may be comprised of multiple separate elements. For example, the fastener may comprise an elongated, threaded body, a separate tie-in-component captured by the elongated portion when emplaced on a vertebral segment and designed to articulate to some extent about the proximal end of the threaded body. The fastener may also comprise a locking mechanism designed to be coupled to the tie-in portion of the fastener with helical threads or by other means. The exemplary configurations described above are given by way of example only, those having familiarity with orthopedic fasteners and spinal fixation devices will recognize numerous fastener and locking mechanism configurations contemplated by the invention.

Fasteners 22, 24 are configured so that their axial path of travel is substantially constrained to the path defined by guides 14, 16. According to some embodiments, the fasteners are cannulated, comprising a centrally located through-bore extending the length of the shaft of the fasteners and open at both the proximal and distal ends of the fastener. The through-bore is sized such that the guides may move relative to the fasteners 22, 24, thus allowing the fasteners to move along the path of guides 14, 16, and also rotate about the axis of the through-bore. Use of such cannulated pedicle screws advantageously allow defining of the eventual path of the fasteners 22, 24 with guides 14, 16 which may be more easily placed using image guided techniques. The narrow gauge of the guides relative to that of the fasteners may also reduce risks inherent in screw placement due to the lessened likelihood of penetrating the pedicle cortex during initial placement of the narrow gauge guides. The path of the guides can then be verified fluoroscopically or otherwise prior to insertion of the cannulated fasteners.

The fasteners 22, 24 may also comprise a driver coupling that allows placement with the aid of a driver configured to engage the driver coupling. Driver coupling may be of any appropriate design including, without limitation, hexagonal interfaces, slotted interfaces, or torx interfaces. Numerous appropriate driver couplings will be apparent to those experienced in the design of drivers and driver interfaces in medical devices and such varied designs are contemplated within the scope of the invention. According to some embodiments, the driver coupling is interrupted by the through-bore of the fastener. In these embodiments, the design of the driver coupling may be chosen such that the through-bore interrupts the driver coupling in a non-torque-bearing location, such as in the center of the cavity of a torx driver interface.

A driver capable of assisting with insertion of the fasteners is also provided according to some embodiments of the present invention. According to some embodiments, the driver interfaces with the driver coupling to assist with insertion of the fastener 22, 24. According to other embodiments, the driver is configured to allow it to interface with the driver coupling when guides 14, 16 are still in position within the through-bore of the fastener before, during, and after emplacement of the fastener. This allows the driver to assist with insertion of the fasteners such as by applying torque while at the same time allowing the guides 14, 16 to remain in place to guide the fasteners 22, 24 into position. As one example, the driver may include a through-bore down its central, or rotational axis sized to allow movement of guide 14, 16 within the through bore relative of the driver. The driver may thus be slid along the path of the guide and into the driver coupling on the fastener 22, 24. The driver may then be rotated about the axis of its through bore, applying torque to the fastener 22, 24 without applying substantial torque to the guide 14, 16.

In some embodiments and under some circumstances, during the emplacement of fasteners 22, 24 guides 14, 16 may be subjected to stresses that result in deformation. When this occurs, the guides 14, 16 may be removed from the spinal segment by backing them out of the through-bore of the fasteners 22, 24. Non-deformed replacement guides may then be inserted back into the spinal segment through the through-bore of the fastener resulting in the replacement guides assuming a path substantially identical to that of the original guides 14, 16.

According to FIG. 1 and FIG. 2 and other embodiments of the invention, the guides 14, 16, fasteners 22, 24, and driver may be inserted into the body, and/or emplaced within a spinal segment using minimally invasive techniques that do not require full exposure of the posterior spinal elements. As one example, these elements may be inserted through small incisions directly above the intended location for placement of the fasteners.

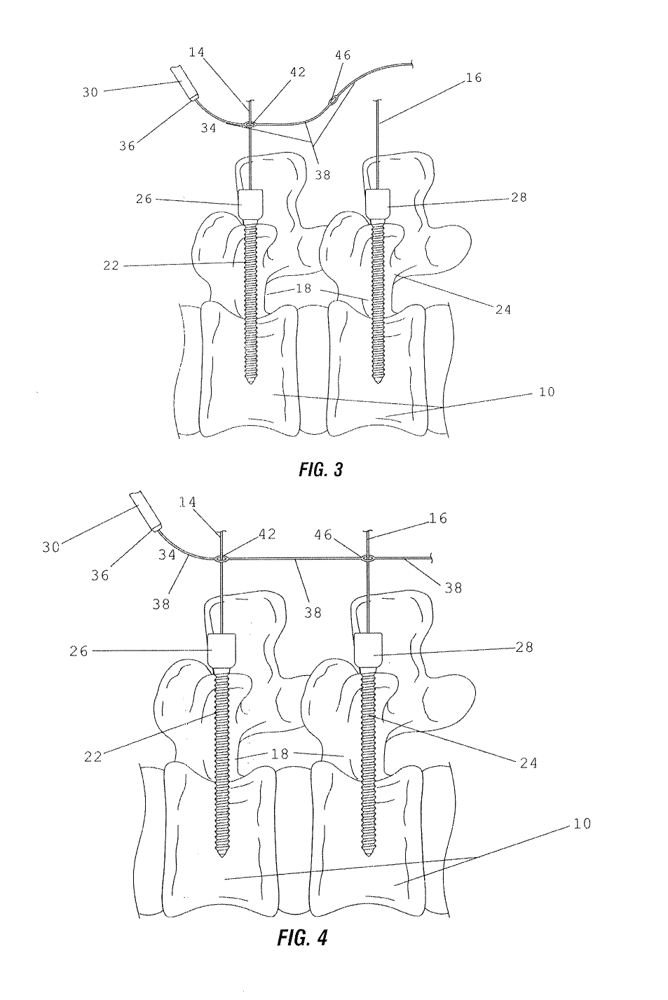

FIG. 3 shows and embodiment of the present invention including the elements of FIG. 2 with the addition of a tie 30 coupled to a tie guide 34. The tie-guide 34 is an elongated guide that is capable of imparting force on the tie 30 sufficient to move it into proximity with fasteners 22, 24, and more specifically into proximity with the tie-in-points 26, 28. According to the embodiment pictured in FIG. 3, the tie rod guide 34 may be comprised of elongated sections 38 coupled to girdling sections 42, 46 which fit around guides 14, 16. These girdling sections may have a ring shaped aperture as depicted in FIG. 3, or may have apertures of any other shape so long as they are capable of girdling, or otherwise fitting around guides 14, 16. In the embodiment depicted, tie 30 is a cylindrical metallic rod having a tapered, or rounded portion 36 disposed near the coupling of the rod 30 and tie guide 34. Tie guide 34 may be inserted into the body via a small incision and extended though the body over each of fasteners 22, 24 with its non-tie-coupled end exiting the body through another small break, or incision in the skin. The embodiment depicted is conducive to minimally invasive emplacement of the tie rod within the body such as through a small incision.

Returning to FIG. 3 girdling section 42 is shown encircling one of the guides 14, 16. The girdling section may be placed into such a position by lifting it posteriorly and positioning it to slide over the end of guide 14, 16 which is not pictured. According to some embodiments, tie guide 34 and girdling section 42 may be lifted posteriorly by flexing the tie guide 34 such that girdling section 42 is extended outside of the body to an end of a guide 14 that is likewise extending posteriorly outside the body. A girdling section may, in some embodiments be extended to fit over a guide 14, 16 through a previously made small incision above a respective fastener 22, 24. Tie guide 34 may be made of a material flexible enough as to allow it to exit the body through such a small incision without excessively damaging surrounding tissues. Tie guide 34 is also preferably long enough that its girdling sections may be extended over wire guides without its free end being pulled back into the body.

FIG. 4 depicts tie-guide 34 after both of its girdling sections 42, 46 have been placed over their respective guides 14, 16.

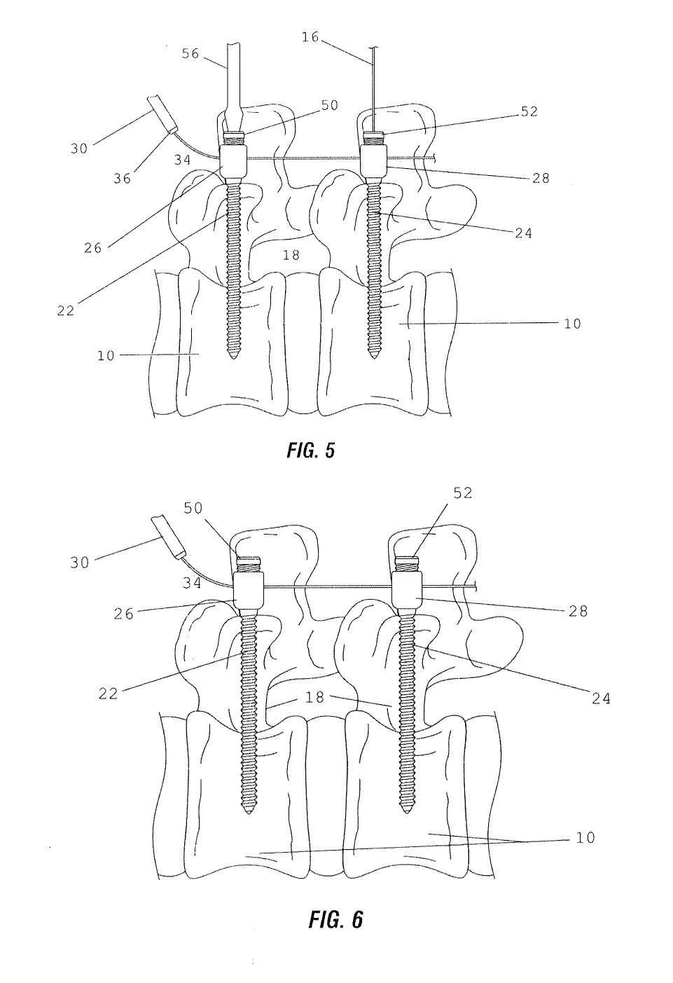

FIG. 5 shows the embodiment of FIG. 4 in which the locking mechanisms 50, 52 of fasteners 22, 24 are partially engaged above respective tie-in points 26, 28, by means of helical threads. Tie guide 34 has also been moved into a position substantially near tie-in points 26, 28 under the locking mechanisms 50, 52. A locking mechanism driver 56 is also depicted. The locking mechanism driver 56 may be the same driver used to engage the driver couplings of fasteners 22, 24 and/or may be similarly designed to engage locking mechanisms 50, 52 while guides 14, 16 are still in place within fasteners 22, 24. In some embodiments locking mechanisms 50, 52 are configured to be partially engaged while guides 14, 16 are still extended through fasteners 22, 24. The locking mechanisms may comprise an axial through-bore sized to allow guides 14, 16 to fit through the locking mechanisms such that the locking mechanisms can rotate axially such as to engage helical threads and otherwise move along the path of the guides. In such embodiments, guides 14, 16 may assist in placement and engagement of the locking mechanisms 50, 52.

Tie guide 34 is moved into a position with portions substantially near the tie-in points 26, 28 with guidance provided by guides 14, 16 by sliding portions of the tie guide 34 and its girdling sections 42, 46 downward with the girdling sections 42, 46 sliding along the path defined by the guides 14, 16. When the tie guide is moved into this position portions of the tie guide between fasteners 22, 24 define a path substantially similar to the desired final orientation of tie 30. According to some embodiments, tie guide 34 may be moved into this position during insertion and partial engagement of the locking mechanisms 50, 52. In some embodiments, the fastener driver may be used to push the tie-guide 34 into position via its action on the locking mechanisms 50, 52. In other embodiments the fastener driver or locking mechanism driver may be used to push the tie-guide 34 into this position prior to partial engagement of the locking mechanisms. Tie guide may also be positioned using any of numerous other surgical instruments that will be apparent to those familiar with surgical techniques and instrumentation. Referring to FIG. 6, once tie guide 34 is in position and locking mechanisms 50, 52 are partially engaged, locking mechanism driver 56 may be removed and guides 14, 16 may be backed out of fasteners 22, 24 and removed from the body.

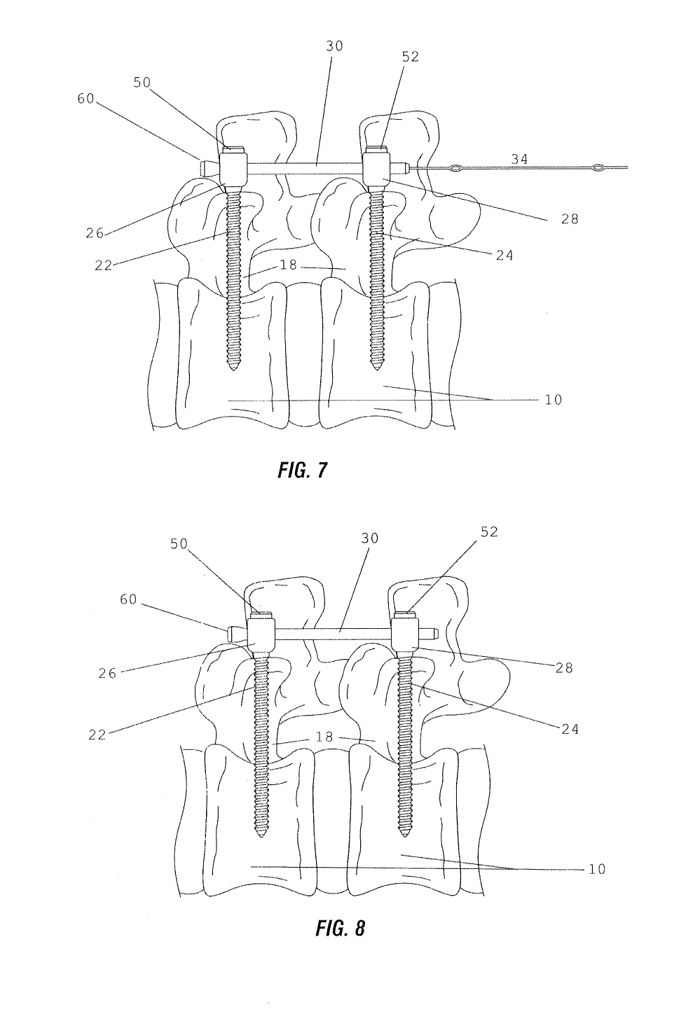

FIG. 7 shows tie guide 34 having pulled tie 30 into position extending between fasteners 22, 24, near their tie-in-points 26, 28, and under locking mechanisms 50, 52. In embodiments where the tie 30 comprises a tapered section, the tapered section may prevent the tie 30 from getting hung up on fasteners 22, 24 while being pulled into position. Tie stopper 60 is shown, having limited movement of tie 30 by butting up against fastener 22. Tie stopper 60 is coupled to tie 30 and has portions extending outside the perimeter of the rest of tie 30 and sized such that the stopper 60 cannot pass through fastener 22 and tie-in-point 26. The stopper 60 may be unitary with the tie, or a separate component coupled to the tie. In some embodiments, tie stopper 60 may be created during, or immediately before a medical procedure by deforming the tie 30 to create a bend, kink, flange or bulge therein. The tie may be configured to permit or accommodate such deformations. Locking mechanisms 50, 52 have been fully engaged to rigidly couple tie 30 to tie-in-points 26, 28.

FIG. 8 shows fully emplaced spine fixation hardware according to one embodiment of the invention. Tie 30 is rigidly affixed to fasteners 22, 24 and has been decoupled from tie guide 34. According to some embodiments tie 30 and tie guide 34 may be decoupled by cutting, grinding or other destructive means. In other embodiments, tie guide 34 may be configured to break away at or near its coupling with tie 30 when sufficient strain is applied. In yet other embodiments, tie guide 34 may be reversibly decouplable from tie 30. Once tie guide 34 is decoupled from tie 30, it may be extracted from the body by pulling on its free end. Incisions above each of the fasteners as well as tie and tie guide incisions may also be closed.

The exemplary embodiments described above and in the Figures extend between two adjacent vertebral segments for illustrative purposes but the invention is not so limited. Any number of adjacent or non-adjacent vertebral segments may be fixed within the scope of the invention. Likewise, multiple fasteners may be inserted into one vertebral segment and/or multiple ties may be affixed to each segment.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1.-23. (canceled)

24. A system for placing orthopedic spine fixation hardware comprising:
an elongated guide;
a tie;
a first orthopedic fastener comprising:
a tie coupling having at least one tie-in-point;
a driver coupling; and
a through-bore along the longitudinal axis of the fastener, the through-bore defining a lumen, the lumen being sized to permit movement of the elongated guide through the lumen;
a tie guide comprising at least one elongated section coupled to at least a one girdling section, the girdling section having an aperture sized to permit movement of the elongated guide through the aperture; and
a driver configured to engage the driver coupling when the elongated guide is disposed through both the lumen and the girdling section;
where the tie is coupled to the tie guide and the tie guide is configured to be capable of pulling at least a portion of the tie into a position substantially near the tie-in-point when the elongated guide is removed from the girdling section.

25. The system of claim 24, further comprising a driver configured to engage the driver coupling when the elongated guide is disposed through both the lumen and the girdling section.

26. The system of claim 25 where the driver is additionally configured to move the girdling section into a position substantially near the tie-in-point.

27. The system of claim 24 where the fastener additionally comprises a locking mechanism, the locking mechanism being configured to engage with the fastener and to couple at least a portion of the tie to the tie-in-point.

28. The system of claim 27 where the locking mechanism is capable of being partially engaged and fully engaged.

29. The system of claim 28 where the tie guide is configured to be capable of pulling at least a portion of the tie into a position substantially near the tie-in-point when the locking mechanism is partially engaged.

30. The system of claim 29 where fully engaging the locking mechanism couples at least a portion of the tie to the tie-in-point.

31. The system of claim 27 where the locking mechanism comprises a through-bore along the longitudinal axis of the fastener, the through-bore defining a passage sized to permit movement of the elongated guide through the passage.

32. The system of claim 31 where the locking mechanism additionally comprises a driver coupling capable of being engaged by the driver when the elongated guide is disposed through the passage in the fastener.

33. The system of claim 24 where the diver comprises a through-bore defining a driver-lumen, the driver-lumen being sized to permit movement of the elongated guide through the driver-lumen.

34. The system of claim 24 where the girdling section is substantially ring-shaped.

35. The system of claim 24 where the elongated guide is a k-wire.

36. The system of claim 35 where the k-wire is a threaded k-wire.

37. The system of claim 24 where the tie-guide is comprised of metallic wire.

38. The system of claim 24 where the orthopedic fastener is a pedicle screw.

39. The system of claim 38 where the tie is a tie rod.

40. The system of claim 39 where the tie rod comprises a stopper.

41. The system of claim 39 where the tie rod comprises at least a portion having a decreased diameter, the portion having a decreased parameter configured to reduce the chance of the tie catching on the pedicle screw.

42. The system of claim 24 where the tie guide is selectively decouplable from the tie.

43. The system of claim 24 where the elongated guide is capable of being removed from the lumen of the orthopedic fastener prior to pulling on the tie-guide.

44. The system of claim 24 comprising two or more orthopedic fasteners.

45. The system of claim 44 where:
the tie guide comprises at least one additional girdling section, the number of girdling sections being equal to the number of orthopedic fasteners;
each of the girdling sections is coupled to another girdling section by an elongated section;
the driver is configured to move each girdling system into a position substantially near each tie in point when engaging the respective driver coupling; and
the tie is coupled to the tie guide and the tie guide is configured to be capable of pulling at least a portion of the tie into a position substantially near each of the tie-in-points.

46. A method comprising:
obtaining a system of claim 24; and
using the system in an orthopedic spinal fixation procedure.
